(19) United States
(12) Patent Application Publication
(10) Pub. No.: US 2022/0257104 A1
(43) Pub. Date: Aug. 18, 2022

(54) ENDOSCOPE WITH A LENS CLEANING NOZZLE

(71) Applicant: OLYMPUS CORPORATION, Tokyo (JP)

(72) Inventors: Ramiya UCHIDA, Tokyo (JP); Takehide KIBAYASHI, Yamato-shi (JP)

(73) Assignee: OLYMPUS CORPORATION, Tokyo (JP)

(21) Appl. No.: 17/665,633

(22) Filed: Feb. 7, 2022

Related U.S. Application Data
(60) Provisional application No. 63/150,147, filed on Feb. 17, 2021.

(51) Int. Cl. A61B 1/12; A61B 1/00; A61B 1/015

(52) U.S. Cl. CPC A61B 1/126; A61B 1/015; A61B 1/00193; A61B 1/00091

(57) ABSTRACT

An endoscope includes an insertion portion coupled to an operation portion and inserted into an object. The insertion portion includes a distal end face, which comprises a first optical system, a second optical system arranged side by side with the first optical system such that a bisector bisecting the first optical system and the second optical system, a first lighting unit, a second lighting unit separate from the first lighting unit so that the first optical system and the second optical system are arranged between the two lighting units, a nozzle portion disposed on a second line at a second predetermined angle with respect to the line perpendicular to the bisector of the first and second optical systems, wherein the nozzle portion is configured to clean the first optical system based on a first cleaning configuration and clean the second optical system based on a second cleaning configuration.

### RELATED APPLICATION DATA

This application is based on and claims priority under 35 U.S.C. § 119 to U.S. Provisional Application No. 63/150,147, filed Feb. 17, 2021, the entire contents of which are incorporated herein by reference.

### FIELD OF THE INVENTION

The present disclosure relates to an endoscope, and more particularly to, a stereoscopic endoscope having a lens cleaning nozzle.

### DESCRIPTION OF THE RELATED ART

In recent years, endoscopes that pick up stereoscopic images of a region to be examined in an object have become popular. Such an endoscope typically includes first and second objective lenses that are respectively provided on a distal end face of an insertion portion of the endoscope. The stereoscopic endoscope also includes first and second image pick up devices, on which the images of a region to be examined are formed respectively via the respective objective lenses. The first and second image pick up devices are provided in a distal end in an inserting direction of the insertion portion.

In an endoscope system, the stereoscopic endoscope is usually connected to an external apparatus, which repeatedly displays, on a monitor, a first observation image formed on the first image pickup device and a second observation image formed on the second image pickup device. An observer observers, via dedicated stereoscopic vision glasses, the first observation image and the second observation image repeatedly displayed on the monitor in such a manner as to observe only the first observation image with one eye and observe only the second observation image with the other eye. As a result, the observer can view a 3D image of the region to be examined in the object.

There has been a desire to make an endoscope that has the distal end face with a smaller diameter. Thus, the endoscope with a single nozzle has been developed. However, such a conventional endoscope with a single nozzle cannot effectively remove water droplets and foreign substances (blood stains or the like) on the objective lenses due to disadvantageous arrangement of the nozzle and the objective lenses.

On the other hand, the endoscope needs to be repeatedly or often cleaned to keep the objective lenses clean. When an insertion portion of the endoscope is inserted into the object, the water droplets and foreign substances such as blood stains or the like may adhere to the lens surfaces or the observation windows for the respective objective lenses provided on the distal end face or fog occurs thereon. In this situation, the region to be examined will be seen differently in the respective observation images. In particular, a 3D image may be impaired even if only a part of one field of view is affected by the water droplets or the foreign substances. Also, the 3D image may be impaired if the nozzle is disposed too close to the objective lenses and is reflected into the field of view.

### SUMMARY OF THE INVENTION

Accordingly, the present disclosure is directed to an endoscope that substantially obviates one or more of the issues due to limitations and disadvantages of related art endoscope.

An object of the present disclosure is to provide an endoscope that includes an operation portion and an insertion portion coupled to the operation portion and inserted into an object to be examined. The insertion portion includes a distal end face, which comprises a first optical system, a second optical system arranged side by side with the first optical system such that a bisector bisecting the first optical system and the second optical system, a first lighting unit, a second lighting unit separate from the first lighting unit so that the first optical system and the second optical system are arranged between the two lighting units, a first channel opening disposed on a first line at a first predetermined angle with respect to a line perpendicular to the bisector of the first and second optical systems, a nozzle portion disposed on a second line at a second predetermined angle with respect to the line perpendicular to the bisector of the first and second optical systems, wherein the nozzle portion is configured to clean the first optical system based on a first cleaning configuration and clean the second optical system based on a second cleaning configuration.

Another object of the present disclosure is to provide an endoscope system that includes the above endoscope for picking up a stereoscopic image of a region to be examined in the object and an image processing unit connected to the stereoscopic endoscope to process the stereoscopic image.

Still another object of the present disclosure is to provide a nozzle for cleaning an endoscope, which comprises a single spout, a first tube connected to the single spout to supply a first fluid to clean a first optical system of the endoscope, a second tube connected to the single spout to supply a second fluid to clean a second optical system of the endoscope, a first pump connected to the first tube to pump the first fluid at a first ejecting rate, and a second pump connected to the second tube to pump the second fluid at a second ejection rate, wherein the first fluid and the second fluid are simultaneously ejected from the single spout.

Still another object of the present disclosure is to provide an endoscope comprises an insertion portion inserted into an object to be examined and including a distal end, the distal end including a distal end face. On the distal end face, a first observation window is arranged for observing a region inside the object; a second observation window is arranged side by side with the first observation window for observing the region inside the object, and having a parallax with respect to the first observation window; a channel is arranged for supplying a fluid to the distal end of the insertion portion from outside the object; and a nozzle is arranged for ejecting the fluid simultaneously toward the first and second observation windows, wherein the nozzle ejects the fluid from the channel into a first ejecting path to clean the first observation window and a second ejecting path to clean the second observation window, and wherein the first ejecting path does not intersect with the second ejecting path.

Additional features and advantages will be set forth in the description that follows, and in part will be apparent from the description, or may be learned by practice of the invention. The objectives and other advantages of the disclosed input device will be realized and attained by the structure particularly pointed out in the written description and claims thereof as well as the appended drawings.

### BRIEF DESCRIPTION OF THE DRAWINGS

The following detailed description of preferred embodiments can be read in connection with the accompanying drawings in which like numerals designate like elements and in which:

FIG. 1 is a diagram schematically showing a configuration of an endoscope system including a stereoscopic endoscope of a first embodiment;

FIG. 2 is a block diagram of the endoscope system shown in FIG. 1;

FIG. 3 is an enlarged plan view of the distal end face of the insertion portion surrounded by an IV line in FIG. 1.

FIG. 4 is the enlarged plan view of the distal end face of the insertion portion surrounded by an IV line in FIG. 1, showing a position relationship between a nozzle and optical systems.

FIG. 5(a) -5(c) are views schematically showing configurations of a nozzle portion according to an exemplary embodiment.

FIGS. 6(a)-6(c) are views schematically showing configurations of a nozzle portion according to another exemplary embodiment.

FIG. 7 is a view schematically showing how fluid is ejected from a nozzle portion according to an exemplary embodiment.

FIGS. 8(a)-8(d) are views schematically showing differently configured spouts suitable for a nozzle portion according to an exemplary embodiment.

Throughout all of the drawings, dimensions of respective constituent elements are appropriately adjusted for clarity. For ease of viewing, in some instances only some of the named features in the figures are labeled with reference numerals.

### DETAILED DESCRIPTION

Exemplary embodiments of the present invention are explained below with reference to the drawings.

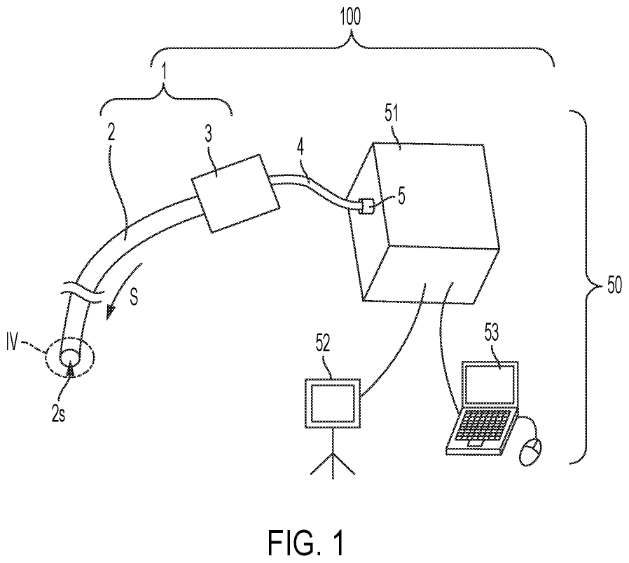

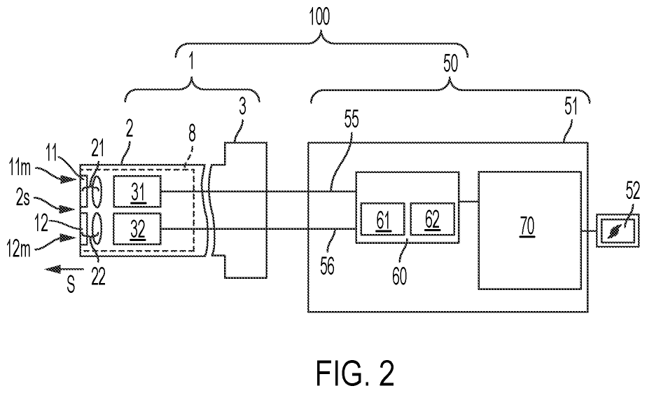

FIG. 1 is a diagram schematically showing a configuration of an endoscope system including an exemplary stereoscopic endoscope. FIG. 2 is a block diagram of the endoscope system shown in FIG. 1. As shown in FIG. 1 and FIG. 2, an endoscope system 100 may include a stereoscopic endoscope 1 that picks up a stereoscopic image of a region to be examined in an object, and peripheral apparatuses 50.

The stereoscopic endoscope 1 may include an insertion portion 2 inserted into the object in an inserting direction S, and an operation portion 3 connected to the insertion portion 2. The stereoscopic endoscope 1 may include a universal cord 4 extended from the operation portion 3 and a connector 5 provided at an extension end of the universal cord 4. The operation portion 3 may include a controller that controls all of operations of the endoscope 1.

As shown in FIG. 1, the stereoscopic endoscope 1 is exemplified by a flexible endoscope in which the insertion portion 2 has flexibility. However, the stereoscopic endoscope 1 may be a rigid endoscope in which the insertion portion 2 is rigid. Alternatively, the insertion portion 2 may include a flexible main portion and a rigid distal end portion having a distal end face 2s.

The peripheral apparatuses 50 may include an image processing unit 51, a display unit 52 such as a 3D monitor connected to the image processing unit 51, and an input unit 53 connected to the image processing unit 51.

The stereoscopic endoscope 1 is connectable to the image processing unit 51 via the connector 5, which may be detachably attached to the image processing unit 51. The stereoscopic endoscope 1 may also be wirelessly connectable to the image processing unit 51 without using the universal cord 4 and the connector 5.

As shown in FIG. 2, an image pickup unit 8 that picks up an image of the region to be examined is provided in a distal end in the inserting direction S of the insertion portion 2. The image pickup unit 8 may include a first objective optical system 21 that observes the region to be examined and a second objective optical system 22 that observes the region to be examined.

The image pickup unit 8 may further include a first image pickup device 31 on which an image of a region to be examined is formed as a first observation image (not shown) via the first objective optical system 21. The image pickup unit 8 may further include a second image pickup device 32 on which an image of the region to be examined is formed as a second observation image (not shown), which has a parallax from the first observation image, via the second objective optical system 22.

In the first objective optical system 21, a lens located front most in the inserting direction S is located as a first objective lens 11 (left eye lens), which is a first observation window, such that a lens surface 11 m is exposed on a distal end face 2s of the distal end of the insertion portion 2. Note that the first observation window is not limited to the objective lens and may be a glass cover or the like. The objective lens is explained as an example of the first observation window below.

In the second objective optical system 22, a lens located front most in the inserting direction S is located as a second objective lens 12 (right eye lens), which is a second observation window, such that a lens surface 12m is exposed on the distal end face 2s. Note that the second observation window is not limited to the objective lens and may be a glass cover or the like. The objective lens is explained as an example of the second observation window below.

Although labeled as left eye lens and right eye lens, such labeling is exemplary and can be exchanged, i.e., first object lens can be a right eye lens and second objective lens can be a left eye lens.

Note that arrangement positions of the first objective lens 11 and the second objective lens 12 on the distal end face 2s and other members provided on the distal end face 2s are explained below.

In the distal end of the insertion portion 2, the first image pickup device 31 may be located behind the first objective optical system 21 in the inserting direction S. Further, in the distal end of the insertion portion 2, the second image pickup device 32 may be located behind the second objective optical system 22 in the inserting direction S.

Referring to FIG. 2, the image processing unit 51 may include a first image pickup cable 55 with a distal end connected to the first image pickup device 31, a second image pickup cable 56 with a distal end connected to the second image pickup device 32, a picked-up image generating section 60, and a stereoscopic display processing section 70.

The first image pickup cable 55 and the second image pickup cable 56 are inserted through the insertion portion 2 and the operation portion 3. When the connector 5 is connected to the image processing unit 51, respective proximal ends of the first image pickup cable 55 and the second image pickup cable 56 are connected to the picked-up image generating section 60 of the image processing unit 51.

The picked-up image generating section 60 may include a first image generating section 61 and a second image generating section 62. The first image generating section 61 generates the first observation image 201 formed on the first image pickup device 31. The second image generating section 62 generates the second observation image 202 formed on the second image pickup device 32.

The picked-up image generating section 60 may be connected to the stereoscopic display processing section 70. Further, the stereoscopic display processing section 70 may be connected to the display unit 52.

The stereoscopic display processing section 70 may alternately repeatedly display the first observation image and the second observation image on the display unit 52. An observer observers, via dedicated stereoscopic vision glasses, the first observation image and the second observation image repeatedly displayed on the display unit 52 in such a manner as to observe only the first observation image with a left eye and observe only the second observation image with a right eye. As a result, the observer can stereoscopically view the region to be examined. In other words, the observer can view a 3D image of the examined region.

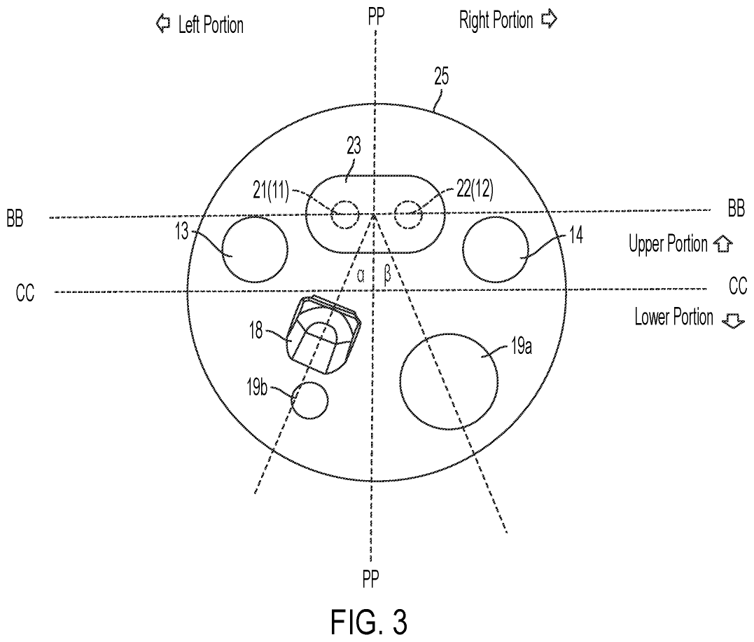

Next, a configuration of the distal end face 2s of the insertion portion 2 is explained with reference to FIG. 3. FIG. 3 is an enlarged plan view of the distal end face 2s of the insertion portion 2 surrounded by an IV line in FIG. 1.

As shown in FIG. 3, the first optical system 21 and the second optical system 22 are provided on the distal end face 2s at the distal end portion of the insertion portion 2. An imaginary line CC passes the center of the distal end face 2s in the left and right direction and corresponds to a diameter that divides the distal end face 2s into two equal portions—an upper portion (UP) and a lower portion (LP). In this exemplary embodiment, the first and second optical systems 21 and 22 may be arranged side by side at the upper portion of the distal end face 2s and spaced apart from each other by a predetermined distance.

The first objective lens 11 of the first optical system 21 may have the same size or the same diameter as the second objective lens 12 of the second optical system 22. The first objective lens 11 of the first optical system 21 may have a different size or a different diameter as the second objective lens 12 of the second optical system 22. In either situation, an imaginary line (L) connecting the centers of the first and second objective lenses 11 and 12 is parallel with the imaginary line CC.

Also, the first optical system 21 and the second optical system 22 may be bisected by a bisector BB into two equal parts, respectively. The bisector BB is an imaginary line parallel with the imaginary line CC. Reference character “PP” denotes an imaginary line perpendicular to the bisector BB of the first and second optical systems 21 and 22. The perpendicular imaginary line PP passes through the center of the distal end face 2s, and divides the distal end face 2s into two equal portions—left portion (lp) and right portion (rp).

The first optical system 21 and the second optical system 22 may be covered by a cover portion 23. The cover portion 23 may be made of a transparent material such as glass or the like. The cover portion 23 may be configured to be detachably attached to the distal end face 2s. In the shown embodiment, the cover portion 23 has a circle shape, but may be a different shape that is suitable for covering the first and second optical systems 21 and 22.

The first optical system 21 is configured to generate the first optical image, and the second optical system 22 is configured to generate the second optical image, such that the first and second optical images may enter with parallax so that the same object image becomes a stereoscopic image.

As shown in FIG. 3, a first light unit 13 and a second light unit 14 are provided on the distal end face 2s and are adjacent to the first and second optical systems 21 and 22, respectively. The first and second light units 13 and 14 are separate by a space sufficient for disposing at least a portion of the first and second optical systems 21 and 22 therebetween. The first and second light units 13 and 14 may be arranged to sandwich the first and second optical systems 21 and 22.

Further on the distal end face 2s, in this exemplary embodiment, a nozzle portion 18 is arranged facing the first and second optical systems 21 and 22 in an oblique direction with respect to the bisector BB. The center line of the nozzle portion 18 may be disposed at a predetermined angle a with respect to the perpendicular imaginary line PP, which is perpendicular to the bisector BB of the first and second optical systems 21 and 22. The predetermined angle a may be in a range of 0 to 60 degrees.

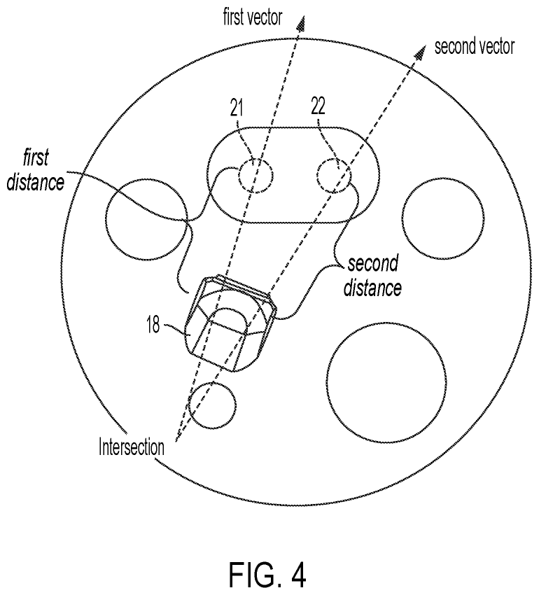

The nozzle portion 18 may be provided at an area of the lower portion of the distal end face 2s, which is close to the first and second optical systems 21 and 22. There is a first distance (see FIG. 4) between the nozzle portion 18 and the first optical system 21, which is set such that the nozzle portion 18 is able to efficiently clean the first optical system 21 without being reflected in the field of view of the first optical system 21. There is a second distance (see FIG. 4) between the nozzle portion 18 and the second optical system 21, which is set such that the nozzle portion 18 is able to efficiently clean the second optical system 22 without being reflected in the field of view of the second optical systems 22. The first distance is different than the second distance.

Further on the distal end face 2s, in this exemplary embodiment, a first channel 19a is arranged facing the first and second optical systems 21 and 22 in an oblique direction with respect to the bisector BB. The center line of the first channel 19a may be positioned at a predetermined angle β with respect to the perpendicular imaginary line PP, which is perpendicular to the bisector BB of the first and second optical systems 21 and 22. The predetermined angle β may be in a range of 0-50 degrees.

The first channel 19a may be provided at the lower portion of the distal end face 2s. The nozzle portion 18 is spaced apart from the first and second optical systems 21 and 22 so that the nozzle portion 18 is not included in the fields of views of both the first and second optical systems 21 and 22. In this exemplary embodiment, the nozzle portion 18 may be disposed at the left portion of the distal end face 2s, and the first channel 19a may be disposed at the right portion of the distal end face 2s. The first channel 19a includes an opening through which an elongated medical device can be inserted into the insertion portion 2 of the endoscope 1 and from which the medical device can protrude to perform medical inspection or medical treatment. The medical devices may be, for example, forceps, stapler, knives, snare, basket, and so on.

Further on the distal end face 2s, in this exemplary embodiment, a second channel 19b may be arranged at a rear side of the nozzle portion 18 with respect to both the first and second optical systems 21 and 22. The second channel 19b may also be disposed at the left portion of the distal end face 2s. The second channel 19b includes an opening through which a fluid is ejected toward to a body tissue of the object to be examined. The opening of the second channel 19b may have a smaller diameter than that of the first channel 19a.

FIG. 4 is the enlarged plan view of the distal end face 2s of the insertion portion 2 surrounded by an IV line in FIG. 1, showing a position relationship between the nozzle portion 18 and the first and second optical systems 21 and 22.

In this exemplary embodiment, the nozzle portion 18 is configured to clean the first optical system 21 based on a first cleaning configuration, and to clean the second optical system 22 based on a second cleaning configuration. The nozzle portion 18 may include a single nozzle, which may be formed with one spout or more than one spouts.

As shown in FIG. 4, the first cleaning configuration of the nozzle portion 18 includes a first vector that indicates a first cleaning direction (or path) toward to the first optical system 21. The second cleaning configuration of the nozzle portion 18 includes a second vector that indicates a second cleaning direction (or path) toward to the second optical system 22. The first and second cleaning directions (paths) have an intersection that is located on a rear side of the nozzle portion 18 with respect to both the first and second optical systems 21 and 22. The first cleaning direction/path is not parallel to the second cleaning direction/path.

The first cleaning configuration of the nozzle portion 18 also includes the first distance from the nozzle portion 18 to the first optical system 21. The second cleaning configuration of the nozzle portion 18 also includes the second distance from the nozzle portion 18 to the second optical system 22. In this exemplary embodiment, the first distance is shorter than the second distance.

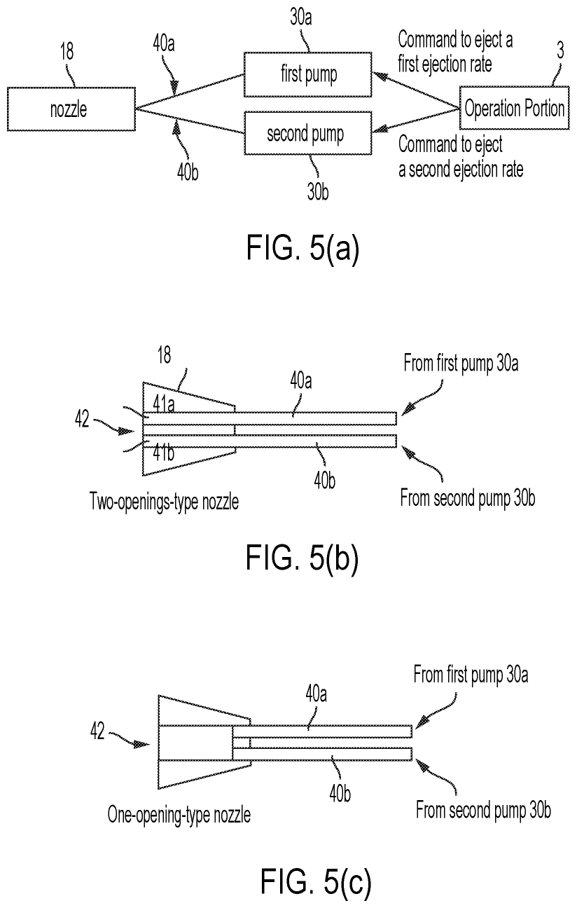

FIG. 5(a) is a view schematically showing the first and second cleaning configurations of the nozzle portion 18 according to an exemplary embodiment. As shown in FIG. 5(a), the first cleaning configuration further includes a first pump 30a connected to the nozzle portion 18 via a first tube 40a. The second cleaning configuration further includes a second pump 30b connected to the nozzle portion 18 via a second tube 40b. The nozzle portion 18 may include an one-opening-type nozzle or a two-openings-type nozzle.

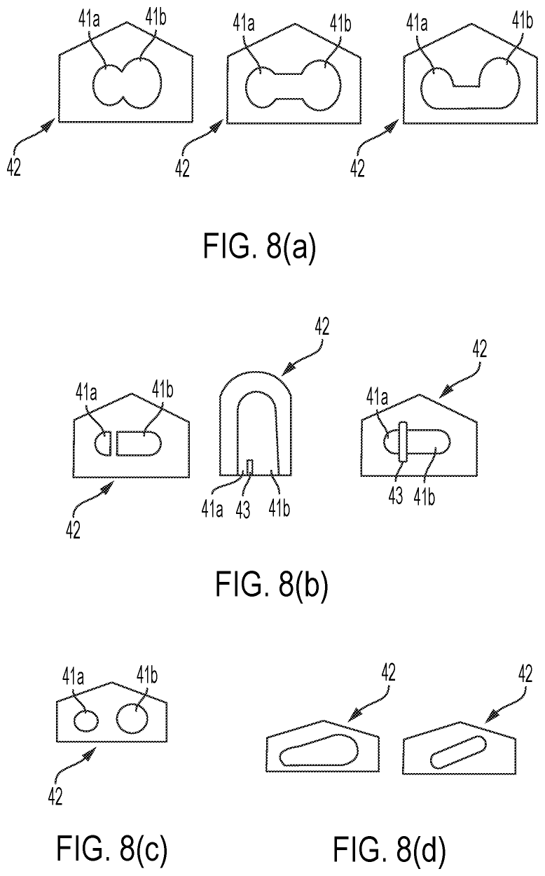

In the two-openings-type nozzle, as shown in FIG. 5(b), the first tube 40a is extended into the nozzle portion 18, and connected to a first opening 41a of a single spout 42 of the nozzle portion 18, and the second tube 40b is extended into the nozzle portion 18, and connected to a second opening 41b of the single spout 42 of the nozzle portion 18. The first opening 41a may be connected with the second opening 41b (see FIG. 8(a)). The first opening 41a and the second opening 41b may be divided by a partition 43 (see FIG. 8(b)). The first opening 41a may be separate from the second opening 41b (see FIG. 8(c)). By this exemplary configuration, the first pump 30a for the first tube 40a and the second pump 30b for the second tube 40b may be activated simultaneously, or the first pump 30a may be activated at a different time than the second pump 30b.

In the one-opening-type nozzle, as shown in FIG. 5C, the first tube 40a and the second tube 40b are extended into the nozzle portion 18, and are merged into one tube inside the nozzle portion 18. In another exemplary embodiment, the first tube 40a and the second tube 40b may be merged inside the insertion portion 2. By this exemplary configuration, the nozzle portion 18 is formed with the single spout 42 having one opening that is connected to both the first tube 4a and the second tube 40b. The first pump 30a for the first tube 40a and the second pump 30b for the second tube 40b may be activated simultaneously, or the first pump 30a may be activated at a different time than the second pump 30b.

When the operation portion 3 sends a command to the first pump 30a, the first pump 30a is activated to supply a fluid at a first ejection rate to the nozzle portion 18 via the first tube 40a, and the nozzle portion 18 thus ejects the fluid and cleans the first optical system 21 at the first ejection rate. When the operation portion 3 sends a command to the second pump 30b, the second pump 30b is activated to supply the fluid at a second ejection rate to the nozzle portion 18 via the second tube 40b, and the nozzle portion 18 thus ejects the fluid and cleans the second optical system 22 at the second ejection rate. The first pump 30a may be activated at a different time than the second pump 30b. The first pump 30a and the second pump 30b may be activated simultaneously.

The fluid may be any one of a liquid, gas, and a mixture of the liquid and the gas.

The first ejection rate may be different from the second ejection rate. In this exemplary embodiment, the first ejection rate is lower than the second ejection rate.

The first tube 40a may have the same diameter as the second tube 40b. The first tube 40a may also have a diameter that is different from that of the second tube 40b.

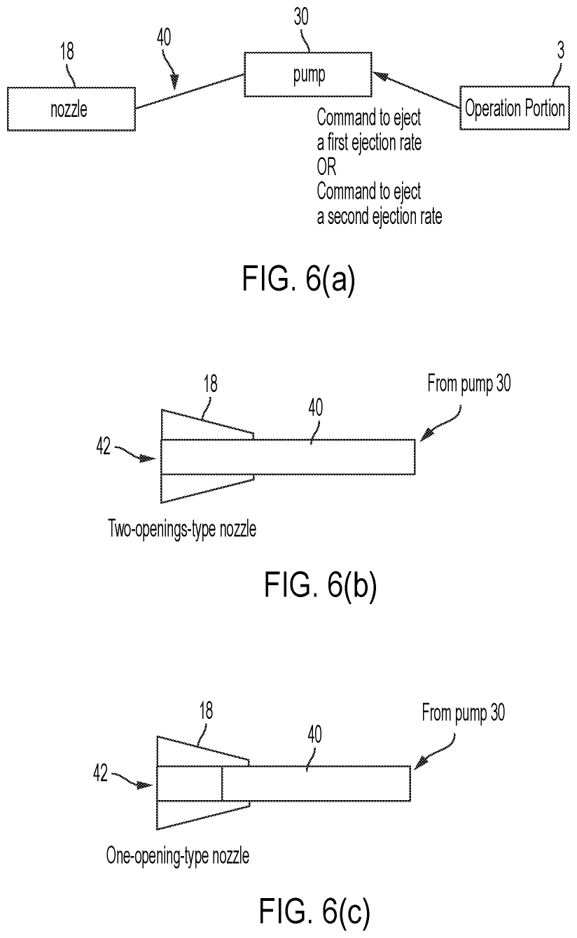

FIG. 6(a) is a view schematically showing the first and second cleaning configurations of the nozzle portion 18 according to another exemplary embodiment. As shown in FIG. 6(a), the first cleaning configuration includes a pump 30 connected to the nozzle portion 18 via a tube 40. In the first cleaning configuration, the fluid is ejected at the first ejection rate. The second cleaning configuration also includes the pump 30 connected to the nozzle portion 18 via the tube 40. In the second cleaning configuration, the fluid is ejected at the second ejection rate. The nozzle portion 18 may include an one-opening-type nozzle or a two-openings-type nozzle.

In the two-openings-type nozzle, as shown in FIG. 6(b), the tube 40 is extended into the nozzle portion 18, and connected to a first opening 41a (in FIGS. 8(a) to 8(c)) of a single spout 42 of the nozzle portion 18, and also connected to a second opening 41b (in FIGS. 8(a) to 8(c)) of the single spout 42 of the nozzle portion 18. The first opening 41a may be connected with the second opening 41b (see FIG. 8(a)). The first opening 41a and the second opening 41b may be divided by a partition 43 (see FIG. 8(b)). The first opening 41a may be separate from the second opening 41b (see FIG. 8(c)). By this exemplary configuration, the pump 30 may be activated to eject the fluid to the first opening 41a and the second opening 41b simultaneously at different ejection rates or the same ejection rate. As shown in FIGS. 8(a) to 8(c), the first opening 41a and the second opening 41b are configured to have different aperture areas, thereby obtaining the different ejection rates. Alternatively, the pump 30 may be activated at one time to eject the fluid to the first opening 41a at the first ejection rate, and at a different time to eject the fluid to the second opening 41b at the second ejection rate.

In the one-opening-type nozzle, as shown in FIG. 6(c), the tube 40 may be formed by merging the first tube 40a and the second tube 40b inside the nozzle portion 18. Alternatively, the tube 40 may be formed by merging the first tube 40a and the second tube 40b inside the insertion portion 2. By this exemplary configuration, the nozzle portion 18 is formed with the single spout 42 as shown in FIG. 8(d). The first ejection rate and the second ejection rate of the pump 30 may be activated alternatively. Moreover, as shown in the left view of FIG. 8(d), the single spout 42 may be configured to have differently sized apertures at right and left end sides of the single spout 42 so as to obtain different ejection rates of the fluid ejected from the different apertures. Also, as shown in the right view of the FIG. 8(d), the single spout 42 is configured to be inclined from a bottom surface thereof, so that the right aperture and the left aperture are set at different heights, thereby further spreading the ejection of the fluid.

When the operation portion 3 sends a first command to the pump 30, the pump 30 is activated to supply a fluid at the first ejection rate to the nozzle portion 18 via the tube 40, and the nozzle portion 18 thus ejects the fluid and cleans the first optical system 21 at the first ejection rate. When the operation portion 3 sends a second command to the pump 30, the pump 30 is activated to supply the fluid at the second ejection rate to the nozzle portion 18 via the tube 40, and the nozzle portion 18 thus ejects the fluid and cleans the second optical system 22 at the second ejection rate. The operation portion may send the first command and the second command at a same time or at a different time. Moreover, even if the pump 30 is activated to supply the fluid under a same pumping pressure, the respective ejection rates of the fluid ejected from the first and second openings 41a and 41b may be different by making the aperture areas of the first and second openings 41a and 41b different.

The fluid may be any one of a liquid, gas, and a mixture of the liquid and the gas.

The first ejection rate may be different from the second ejection rate. In this exemplary embodiment, the first ejection rate is lower than the second ejection rate.

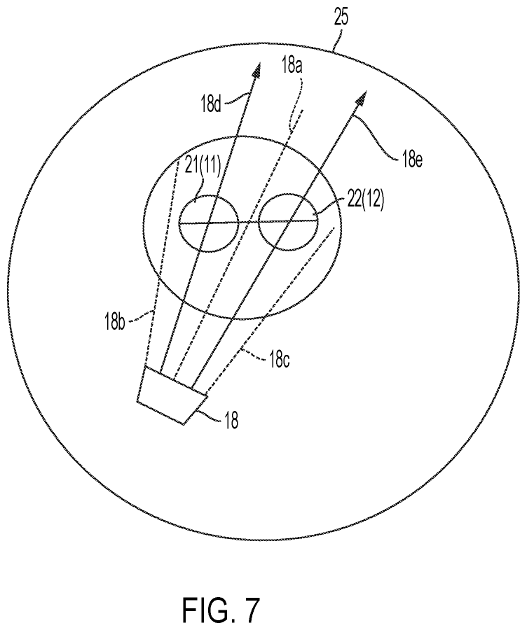

FIG. 7 is a view schematically showing the first and second ejecting configurations according to another exemplary embodiment. As shown in FIG. 7, the nozzle portion 18 is arranged in an oblique angle with respect to the first and second optical systems 21(11) and 22(12). The first cleaning configuration includes a first ejecting region FR, and the second cleaning configuration includes a second ejecting region SR. The first ejection region FR and the second ejecting region SR are divided by a center line 18a of the nozzle portion 18 (or the nozzle injecting direction).

In this exemplary embodiment, it is defined that, in the front view of the distal end face 2s of the endoscope, the left side 21 (11) is the right eye and the right side 22 (12) is the left eye.

The center line 18a may be positioned extending in a direction of passing an area between the first optical system 21 and the second optical system 22. The center line 18a may slightly overlap either the first optical system 21 or the second optical system 22. However, the center line 18a is positioned not to pass or overlap both the first optical system 21 and the second optical system 22. In particular, the center line 18a is positioned not to overlap both the first objective lens 11 (the right eye lens) of the first optical system 21 and the second objective lens 12 (the left eye lens) in the second optical system 22.

By such a configuration, the nozzle portion 18 is prevented from being substantially arranged in the same direction on which the first optical system 21 and the second optical system 22 are arranged on the distal end face 2s, because it becomes difficult for a nozzle to remove stains or the like remaining at a place far from the nozzle as the flow rate (or pressure) of the ejected fluid lowers as a function of distance from the nozzle. In other words, when the center line 18a of the nozzle injecting direction passes through both the right eye lens 12 and the left eye lens 11, it is difficult to simultaneously clean the long paths of the left and right field of view (horizontal of the left eye image+horizontal of the right eye image).

In the configuration of this embodiment, the path (vertical of the left eye image and vertical of the right eye image) can be shortened, and the time until water droplets, dirt, etc. pass can be shortened.

Referring to FIG. 7, the first ejecting region FR may be defined by the center line 18a and a side line 18b. The second ejecting region SR may be defined by the center line 18a and a side line 18c. The side line 18b is positioned extending from the nozzle portion 18 toward the first optical system 21 in a direction away from the center line 18a. The side line 18c is positioned extending from the nozzle portion 18 toward the second optical system 22 in a direction away from the center line 18a. Thus, the first ejecting region FR may be shaped as a taper and gradually increases its size toward the first optical system 21, so that the first ejecting region fully covers the first optical system 21. In exemplary embodiment, the first ejecting region FR does not overlap with the second objective lens 12 of the second optical system 22. In another exemplary embodiment, the first ejection region FR may slightly overlap with the second objective lens 12 of the second optical system 22. The second ejecting region SR may also be shaped as a taper and gradually increases its size toward the second optical system 21, so that the second ejecting region SR fully covers the second optical system 22. In exemplary embodiment, the second ejecting region SR does not overlap with the first objective lens 11 of the first optical system 21. In another exemplary embodiment, the second ejection region SR may slightly overlap with the first objective lens 11 of the first optical system 21.

Moreover, as shown in FIG. 7, the first ejecting region has a center line 18d extending toward the first optical system 21, and the second ejecting region has a center line 18e extending toward the second optical system 22. The center line 18d is not parallel to the center line 18e. The center line 18d and the center line 18e extend to be an opening angle. By this exemplary configuration, the fluid ejected by the first cleaning configuration effectively cleans the first optical system 21 within the first ejecting region, and the fluid ejected by the second cleaning configuration effectively cleans the second optical system 22 within the second ejecting region. As a result, the water droplets, stains or the like flowing out of one field of view can be prevented from flowing to the other field of view, thereby reducing the time for the water droplets, stains or the like remaining on the other field of view.

FIGS. 8(a)-8(d) are views schematically showing differently configured spouts suitable for the nozzle portion 18 as exemplary embodiments. FIG. 8(a) illustrates three (3) exemplary patterns suitable for the spout 42 of the nozzle portion 18. The single spout 42 includes two circles that are connected to each other. The left circle (smaller one) corresponds to the first opening 41a, and the right circle (larger one) corresponds to the second opening 41b. As shown by the left exemplary pattern of FIG. 8(a), the spout 42 may be shaped like two circles having different diameters slightly overlapping with each other. As shown by the middle exemplary pattern of FIG. 8(a), the spout 42 may be shaped like two circles having different diameters connected to each other via a shared space such as a parallelogram-shaped space. The right exemplary pattern of FIG. 8(a) is similar to the middle exemplary pattern of FIG. 8(a) except that the right exemplary pattern of FIG. 8(a) includes a flat bottom surface joining the two circles (the first opening 41a and the second opening 41b) as the shared space.

FIG. 8(b) illustrates other different exemplary patterns suitable for a spout 42 of the nozzle portion 18. As the left exemplary pattern of FIG. 8(b), the spout 42 may be divided into two different parts by a partition 43. The left part (smaller one) corresponds to the first opening 40a, and the right part (larger one) corresponds to the second opening 40b. The middle view of FIG. 8(b) is a cross-sectional view V-V of the left exemplary pattern showing an integrally formed partition 43. The right exemplary pattern of FIG. 8(b) shows that the spout 42 may be divided into two different parts (the first opening 41a and the second opening 41b) by a partition 43 that is detachably attached to the spout.

FIG. 8(c) illustrates a further different exemplary pattern suitable for a spout 42 suitable for the nozzle portion 18. As shown in FIG. 8(c), the spout 42 may be formed by two separate openings, which correspond to the first opening 41a and the second opening 41b, and are connected to the first tube 40a and the second tube 40b, respectively.

FIG. 8(d) illustrates further different exemplary patterns suitable for a spout 42 suitable for the nozzle portion 18. The single spout 42 is configured to be connected to both thefirst tube 40a and the second tube 40b. As the left exemplary pattern of FIG. 8(d), the spout 42 may be shaped like a cone that includes two differently sized end portions. As the right exemplary pattern of FIG. 8(d), the spout 42 may be shaped like an inclined disposed rectangular or other geometric shape with two end portions—preferably curved portions.

Although the present invention has been described in connection with the above exemplary embodiments, it will be appreciated by those skilled in the art that additions, deletions, modifications, and substitutions not specifically described may be made without department from the spirit and scope of the invention as defined in the appended claims.

What is claimed is:

1. An endoscope, comprising:
an operation portion; and
an insertion portion coupled to the operation portion and configured to be inserted into an object to be examined, the insertion portion including a distal end face,
the distal end face of the insertion portion comprising:
a first optical system,
a second optical system arranged side by side with the first optical system such that a bisector bisecting the first optical system and the second optical system,
a first lighting unit,
a second lighting unit separate from the first lighting unit so that the first optical system and the second optical system are arranged between the two lighting units,
a first channel opening disposed on a first line at a first predetermined angle with respect to a line perpendicular to the bisector of the first and second optical systems, and
a nozzle portion disposed on a second line at a second predetermined angle with respect to the line perpendicular to the bisector of the first and second optical systems, wherein the nozzle portion is configured to:
clean the first optical system based on a first cleaning configuration, and
clean the second optical system based on a second cleaning configuration.

2. The endoscope according to claim 1, wherein the distal end face is further comprising:
a cover portion covering both the first optical system and the second optical system, the cover portion including a first region corresponding to the first optical system and a second region corresponding to the second optical system,
wherein the first cleaning configuration is configured to clean the first region of the cover portion, and the second cleaning configuration is configured to clean the second region of the cover portion.

3. The endoscope according to claim 1, wherein the first and second cleaning configurations are configured to simultaneously clean the first and second optical systems, respectively.

4. The endoscope according to claim 3, wherein the nozzle portion comprises a nozzle including a single spout, and the nozzle portion is configured to eject a fluid out of the single spout to clean both the first optical system and the second optical system.

5. The endoscope according to claim 1, wherein the nozzle portion comprises a single nozzle, the single nozzle including:
a first opening configured to eject a fluid toward to the first optical system based on the first cleaning configuration, and
a second opening configured to eject a fluid toward to the second optical system based on the second cleaning configuration.

6. The endoscope according to claim 1, wherein the first cleaning configuration includes a first vector indicating a first cleaning direction toward the first optical system,
the second cleaning configuration includes a second vector indicating a second cleaning direction toward to the second optical system, and
the first vector intersects with the second vector at an intersection that is located on a rear side of the nozzle portion with respect to both the first optical system and the second optical system.

7. The endoscope according to claim 1, wherein the first cleaning configuration includes a first distance that is set from the nozzle portion to the first optical system,
the second cleaning configuration includes a second distance that is set from the nozzle portion to the second optical system, and
the first distance is shorter than the second distance.

8. The endoscope according to claim 1, wherein the distal end face further comprises a second channel opening, through which a fluid is ejected toward a body tissue of the object, and
the second channel opening is disposed on a rear side of the nozzle portion with respect to both the first optical system and the second optical system.

9. The endoscope according to claim 8, wherein the first channel opening is configured to protrude an elongated medical device inserted into the insertion portion from a proximal side of the endoscope.

10. The endoscope according to claim 1, wherein the first cleaning configuration includes a first opening and the second cleaning configuration includes a second opening, and
wherein the first opening is different from the second opening in size.

11. The endoscope according to claim 1, wherein the first cleaning configuration is configured to eject a fluid at a first ejection rate, and the second cleaning configuration is configured to eject the fluid at a second ejection rate, and
wherein the first ejection rate is different from the second ejection rate.

12. The endoscope according to claim 1, wherein the nozzle portion comprises
a single spout;
a first tube connected to the single spout to supply a first fluid to clean the first optical system;
a second tube connected to the single spout to supply a second fluid to clean the second optical system;
a first pump connected to the first tube to pump the first fluid at a first ejecting rate; and
a second pump connected to the second tube to pump the second fluid at a second ejection rate,
wherein the first fluid and the second fluid are simultaneously ejected from the single spout.

13. The endoscope according to claim 12, wherein the first ejecting rate is different from the second ejecting rate.

14. The endoscope according to claim 13, wherein the single spout includes a first opening configured to eject the first fluid toward to the first optical system and a second opening configured to eject the second fluid toward the second optical system.

15. The endoscope according to claim 14, wherein the first opening is different from the second opening in size.

16. The endoscope according to claim 14, wherein the first opening is connected to the second opening.

17. The endoscope according to claim 14, wherein the first opening is separate from the second opening by a partition.

18. The endoscope according to claim 14, further includes a partition that is detachably attached to the single spout to separate the first opening and the second opening.

19. An endoscope system, comprising:
an endoscope according to claim 1 for picking up a stereoscopic image of a region to be examined in the object; and
an image processing unit connected to the stereoscopic endoscope to process the stereoscopic image.

20. The endoscope system according to claim 19, further comprising:
a display unit for displaying an image processed by the image processing unit; and
an input unit connected to the image processing unit.

21. An endoscope, comprising:
an insertion portion configured to be inserted into an object to be examined and including a distal end, the distal end including a distal end face,
on the distal end face:
a first observation window arranged for observing a region inside the object,
a second observation window arranged side by side with the first observation window for observing the region inside the object, and having a parallax with respect to the first observation window,
a channel arranged for supplying a fluid to the distal end of the insertion portion from outside the object, and
a nozzle arranged for ejecting the fluid simultaneously toward the first and second observation windows,
wherein the nozzle ejects the fluid from the channel into a first ejecting path to clean the first observation window and a second ejecting path to clean the second observation window, and
wherein the first ejecting path does not intersect with the second ejecting path.

22. The endoscope according to claim 21, wherein a center line of the first ejecting path is not parallel to a center line of the second ejecting path.

23. The endoscope according to claim 21, wherein the nozzle includes a single spout from which the first ejecting path and the second ejecting path are formed.

24. The endoscope according to claim 23, wherein the single spout includes a first opening for the first ejecting path and a second opening for the second ejecting path, and
wherein the first opening is connected to the second opening.

25. The endoscope according to claim 24, wherein the first opening is different from the second opening in size.

26. The endoscope according to claim 23, wherein the single spout includes a partition that is detachably attached to the spout and divides the spout into a first opening for the first ejecting path and a second opening for the second ejecting path.
